(19) United States
(12) Patent Application Publication
(10) Pub. No.: US 2005/0123091 A1
(43) Pub. Date: Jun. 9, 2005

(54) Three-dimensional backprojection method and apparatus, and X-ray CT apparatus

(76) Inventors: Akihiko Nishide, Tokyo (JP); Akira Hagiwara, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(21) Appl. No.: 11/009,751

(22) Filed: Dec. 10, 2004

(30) Foreign Application Priority Data
Dec. 9, 2003 (JP) 2003-410835
Dec. 10, 2003 (JP) 2003-411283

(51) Int. Cl. G21K 1/12; H05G 1/60; A61B 6/00; G01N 23/00

(52) U.S. Cl. 378/19

(57) ABSTRACT

A method for image reconstruction in an X-ray CT apparatus, wherein projection data D0 collected by an axial scan using a multi-row X-ray detector or planar X-ray detector having a plurality of detectors is plane-projected onto a projection plane to determine plane-projected data D1; then the plane-projected data D1 is projected in a direction of X-ray transmission onto pixels constituting a plurality of lines arranged successively in a direction parallel to a projection plane at spacings of a plurality of pixels on a reconstruction field, to determine backprojected pixel data D2 for pixels constituting lines on the reconstruction field for a number of plane-projected data lines that depends upon the angle formed between the plane of the reconstruction field and X-ray beam; and the plurality of lines are interpolated to determine backprojected pixel data D2 for pixels in between the lines on the reconstruction field.

### BACKGROUND OF THE INVENTION

The present invention relates to a three-dimensional backprojection method and apparatus, and an X-ray CT (computed tomography) apparatus, and to image reconstruction according to a three-dimensional backprojection method in a conventional scan (axial scan).

In conducting image reconstruction for a conventional scan (axial scan) based on a three-dimensional backprojection method, processing is conducted for each row in a multi-row detector by an image reconstruction method common to the rows (for example, see Patent Document 1).

[Patent Document 1]Japanese Patent Application Laid Open No. 2003-225230 (pages 9-10, FIGS. 1-2)

The image reconstruction based on the three-dimensional backprojection method generally poses a problem in that it requires a larger amount of calculation than that of image reconstruction based on two-dimensional backprojection, and is more time-consuming.

### SUMMARY OF THE INVENTION

It is therefore an object of the present invention to provide a three-dimensional backprojection method and apparatus that requires a smaller amount of calculation and is less time-consuming, and an X-ray CT apparatus comprising such a three-dimensional backprojection apparatus.

(1) The present invention, in one aspect for solving the aforementioned problem, is a three-dimensional backprojection method characterized in comprising: plane-projecting projection data D0 collected by a conventional scan (axial scan) using a multi-row X-ray detector or planar X-ray detector having a plurality of detectors, onto a projection plane to determine plane-projected data D1; then projecting said plane-projected data D1 in a direction of X-ray transmission onto pixels constituting a plurality of lines arranged successively in a direction parallel to the projection plane at spacings of a plurality of pixels on a reconstruction field, to determine backprojected pixel data D2 for pixels constituting lines on the reconstruction field for a number of plane-projected data lines that depends upon the angle formed between the plane of the reconstruction field and X-ray beam; interpolating said plurality of lines to determine backprojected pixel data D2 for pixels in between the lines on the reconstruction field; and adding the backprojected pixel data D2 on a pixel-by-pixel basis for all views used in image reconstruction to determine backprojected data D3.

(2) The present invention, in another aspect for solving the aforementioned problem, is a three-dimensional backprojection apparatus characterized in comprising: plane-projected data calculating means for plane-projecting projection data D0 collected by a conventional scan (axial scan) using a multi-row X-ray detector or planar X-ray detector having a plurality of detectors, onto a projection plane to determine plane-projected data D1; backprojected pixel data calculating means for projecting said plane-projected data D1 in a direction of X-ray transmission onto pixels constituting a plurality of lines arranged successively in a direction parallel to the projection plane at spacings of a plurality of pixels on a reconstruction field, to determine backprojected pixel data D2 for pixels constituting lines on the reconstruction field for a number of plane-projected data lines that depends upon the angle formed between the plane of the reconstruction field and X-ray beam, and for interpolating in between said plurality of lines to determine backprojected pixel data D2 for pixels in between the lines on the reconstruction field; and backprojected data calculating means for adding the backprojected pixel data D2 on a pixel-by-pixel basis for all views used in image reconstruction to determine backprojected data D3.

(3) The present invention, in still another aspect for solving the aforementioned problem, is an X-ray CT apparatus characterized in comprising: an X-ray tube; a multi-row detector having a plurality of detector rows; scanning means for collecting projection data D0 while rotating at least one of said X-ray tube and said multi-row detector around a subject to be imaged; plane-projected data calculating means for determining plane-projected data D1 plane-projected onto a projection plane based on said projection data D0; backprojected pixel data calculating means for projecting said plane-projected data D1 in a direction of X-ray transmission onto pixels constituting a plurality of lines arranged successively in a direction parallel to the projection plane at spacings of a plurality of pixels on a reconstruction field, to determine backprojected pixel data D2 for pixels constituting lines on the reconstruction field for a number of plane-projected data lines that depends upon the angle formed between the plane of the reconstruction field and X-ray beam, and for interpolating in between said plurality of lines to determine backprojected pixel data D2 for pixels in between the lines on the reconstruction field; and backprojected data calculating means for adding the backprojected pixel data D2 on a pixel-by-pixel basis for all views used in image reconstruction to determine backprojected data D3.

Preferably, the number of plane-projected data lines is optimized taking image quality of an image to be reconstructed into account, so that the amount of calculation can be optimized according to target image quality.

Preferably, representing a direction perpendicular to a plane of rotation of the X-ray tube and X-ray detector as z-direction, a direction of the center axis of the X-ray beam at a rotation angle of 0° as y-direction, and a direction orthogonal to the z- and y-directions as x-direction, said projection plane is defined as the x-z plane that passes through a center of rotation for −45°≦rotation angle<45°or a rotation angle range mainly including the range and also including its vicinity and 135°≦rotation angle<225° or a rotation angle range mainly including the range and also including its vicinity, and said projection plane is defined as the y-z plane that passes through the center of rotation for 45°≦rotation angle<135° or a rotation angle range mainly including the range and also including its vicinity and 225° ≦rotation angle<315° or a rotation angle range mainly including the range and also including its vicinity, so that the plane-projected data D1 can be appropriately determined.

Preferably, each data element of the plane-projected data D1 is determined from a plurality of data elements of the projection data D0 by extrapolation, so that the plane-projected data D1 can be appropriately determined. Preferably, each data element of the backprojected pixel data D2 is determined by weighted addition on a plurality of data elements of the plane-projected data D1, so that the backprojected pixel data D2 can be appropriately determined.

Preferably, the backprojected pixel data D2 is determined as the result of weighted addition on backprojected pixel data D2 at a certain rotation angle (view) and backprojected pixel data D2 at an opposite rotation angle (view) with weighting factors wₐ and w_{b} (wₐ+w_{b}=1) that depend upon the angle formed by a straight line connecting a pixel on the reconstruction field at these views and an X-ray focal spot with respect to the plane of the reconstruction field, so that the backprojected pixel data D2 can be appropriately determined.

In the invention described in (1)-(3), the plane-projected data D1 is projected in a direction of X-ray transmission onto pixels constituting a plurality of lines arranged successively in a direction parallel to the projection plane at spacings of a plurality of pixels on a reconstruction field, to determine backprojected pixel data D2 for pixels constituting lines on the reconstruction field for a number of plane-projected data lines that depends upon the angle formed between the plane of the reconstruction field and X-ray beam, and therefore, the number of plane-projected data lines is decreased for a smaller angle between the plane of the reconstruction field and X-ray beam, thus reducing the amount of calculation.

Specifically, according to the present invention, there is provided a three-dimensional backprojection method and apparatus and an X-ray CT apparatus characterized in that they control or optimize the time for reconstruction by controlling or optimizing the number of lines for planar projection according to the cone angle formed between the reconstruction plane (X-Y plane) and X-ray beam.

According to the three-dimensional backprojection method and apparatus and X-ray CT apparatus of the present invention, the time for reconstruction is reduced by decreasing the number of lines for planar projection for a smaller cone angle formed between the reconstruction plane (X-Y plane) and X-ray beam, and increasing it for a larger cone angle.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing an X-ray CT apparatus in accordance with one embodiment of the present invention.

FIG. 2 is an explanatory diagram showing a rotation of an X-ray tube and a multi-row detector.

FIG. 3 is an explanatory diagram showing a cone beam.

FIG. 4 is a flow chart showing a general operation of the X-ray CT apparatus in accordance with one embodiment of the present invention.

FIG. 5 is a flow chart showing details of three-dimensional image reconstruction processing.

FIG. 6 is a conceptual diagram showing lines on a reconstruction field projected in the direction of X-ray transmission.

FIG. 7 is a conceptual diagram showing lines projected onto a detector plane.

FIG. 8 is a conceptual diagram showing projection data Dr on lines at a rotation angle=0° projected onto a projection plane.

FIG. 9 is a conceptual diagram showing projection line data Dp on the lines at the rotation angle=0° projected onto the projection plane.

FIG. 10 is a conceptual diagram showing image-positional line data Df on the lines at the rotation angle=0° projected onto the projection plane.

FIG. 11 is a conceptual diagram showing high density image-positional line data Dh on the lines at the rotation angle=0° projected onto the projection plane.

FIG. 12 is a conceptual diagram showing backprojected pixel data D2 on lines on a reconstruction field at the rotation angle=0°.

FIG. 13 is a conceptual diagram showing backprojected pixel data D2 of pixels on the reconstruction field at the rotation angle=0°.

FIG. 14 is a conceptual diagram showing projection data Dr on lines at a rotation angle=90° projected onto a projection plane.

FIG. 15 is a conceptual diagram showing projection line data Dp on the lines at the rotation angle=90° projected onto the projection plane.

FIG. 16 is a conceptual diagram showing image-positional line data Df on the lines at the rotation angle=90° projected onto the projection plane.

FIG. 17 is a conceptual diagram showing high density image-positional line data Dh on the lines at the rotation angle=90° projected onto the projection plane.

FIG. 18 is a conceptual diagram showing backprojected pixel data D2 on lines on a reconstruction field at the rotation angle=90°.

FIG. 19 is a conceptual diagram showing backprojected pixel data D2 of pixels on the reconstruction field at the rotation angle=90°.

FIG. 20 is an explanatory diagram showing backprojected data D3 obtained by adding the backprojected pixel data D2 on a pixel-by-pixel basis for all views.

FIG. 21 is an explanatory diagram showing that sufficient image quality can be attained for a small cone angle even if the number of planar projection lines is small.

### DETAILED DESCRIPTION OF THE INVENTION

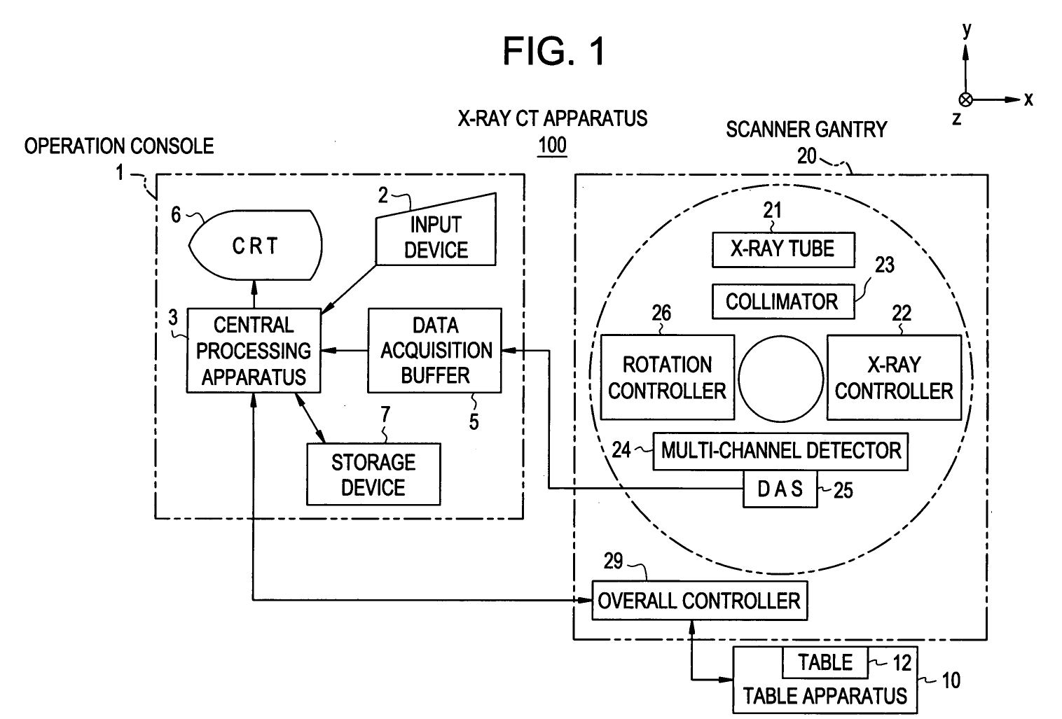

The best mode for carrying out the present invention will now be described in detail with reference to the accompanying drawings. It should be noted that the present invention is not limited to the best mode for carrying out the present invention. FIG. 1 shows a block diagram of an X-ray CT apparatus. The present apparatus is an example of the best mode for carrying out the present invention. The configuration of the present apparatus represents an example of the best mode for carrying out the present invention with respect to the three-dimensional backprojection apparatus or X-ray CT apparatus of the present invention. The operation of the present apparatus represents an example of the best mode for carrying out the present invention with respect to the three-dimensional backprojection method of the present invention.

The X-ray CT apparatus 100 comprises an operation console 1, an imaging table 10, and a scan gantry 20. The operation console 1 comprises an input device 2 for accepting inputs by a human operator, a central processing apparatus 3 for executing three-dimensional backprojection processing in accordance with the present invention etc., a data collection buffer 5 for collecting projection data acquired at the scan gantry 20, a CRT 6 for displaying a CT image reconstructed from the projection data, and a storage device 7 for storing programs, data, and X-ray CT images.

The table apparatus 10 comprises a cradle 12 for laying thereon a subject and transporting the subject into/out of a bore (cavity portion) of the scan gantry 20. The cradle 12 is driven by a motor built in the table apparatus 10.

The scan gantry 20 comprises an X-ray tube 21, an X-ray controller 22, a collimator 23, a multi-row detector 24, a DAS (data acquisition system) 25, a rotation controller 26 for rotating the X-ray tube 21 and the like around the body axis of the subject, and a control interface 29 for communicating control signals etc. with the operation console 1 and imaging table 10. Also via the control interface 29, the X-ray controller 22, collimator 23 and rotation controller 26 are controlled by the central processing apparatus 3.

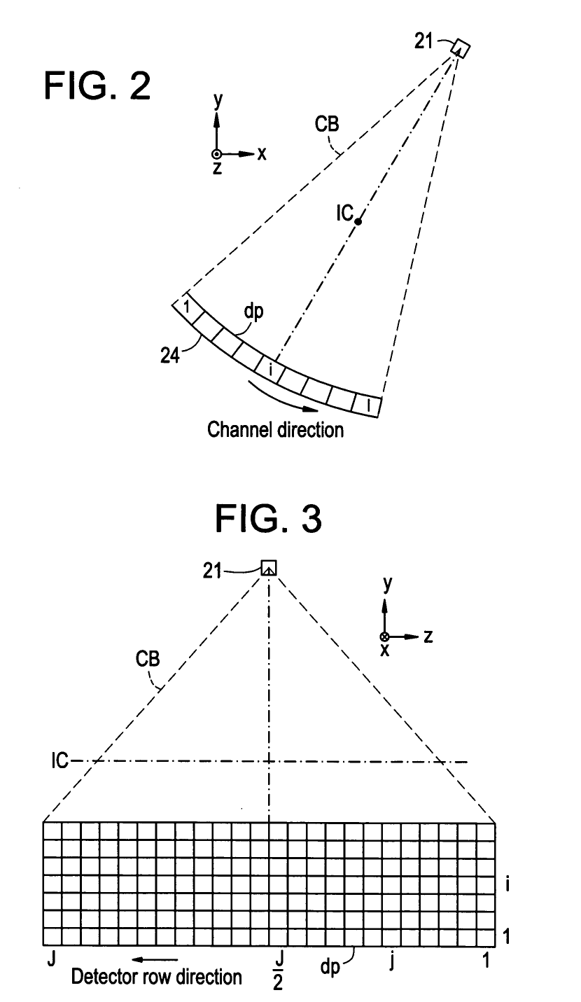

The following description will be made on a conventional scan (axial scan). FIGS. 2 and 3 are explanatory diagrams of the X-ray tube 21 and multi-row detector 24. The X-ray tube 21 and multi-row detector 24 rotate around a center of rotation IC. Representing the vertical direction as y-direction, the horizontal direction as x-direction, and a direction perpendicular to these directions as z-direction, the plane of rotation of the X-ray tube 21 and multi-row detector 24 is the x-y plane. The direction of translation of the cradle 12 is the z-direction. In place of the multi-row detector 24, a planar X-ray detector may be employed.

The X-ray tube 21 generates an X-ray beam generally referred to as cone beam CB. The direction of the center axis of the cone beam CB parallel to the y-direction is defined as a rotation angle=0°. The multi-row detector 24 has 256 detector rows, for example. Each detector row has 1,024 channels, for example.

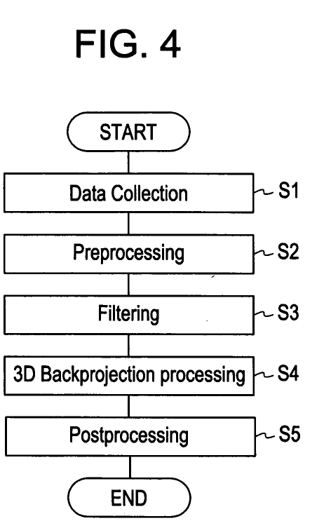

FIG. 4 is a flow chart showing the general operation of the X-ray CT apparatus 100. At Step S1, projection data D0(z, view, j, i) represented by the table's rectilinear motion position z, view angle view, detector row index j and channel index i is collected while rotating the X-ray tube 21 and multi-row detector 24 around the subject to be imaged. The data collection is conducted by the scan gantry 20. The scan gantry 20 is an example of the scanning means of the present invention

At Step S2, the projection data D0(z, view, j, i) is subjected to pre-processing (offset correction, log correction, X-ray dose correction and sensitivity correction). At Step S3, the pre-processed projection data D0(z, view, j, i) is filtered. Specifically, the data is subjected to Fourier transformation, multiplied by a filter (reconstruction function), and then subjected to inverse Fourier transformation.

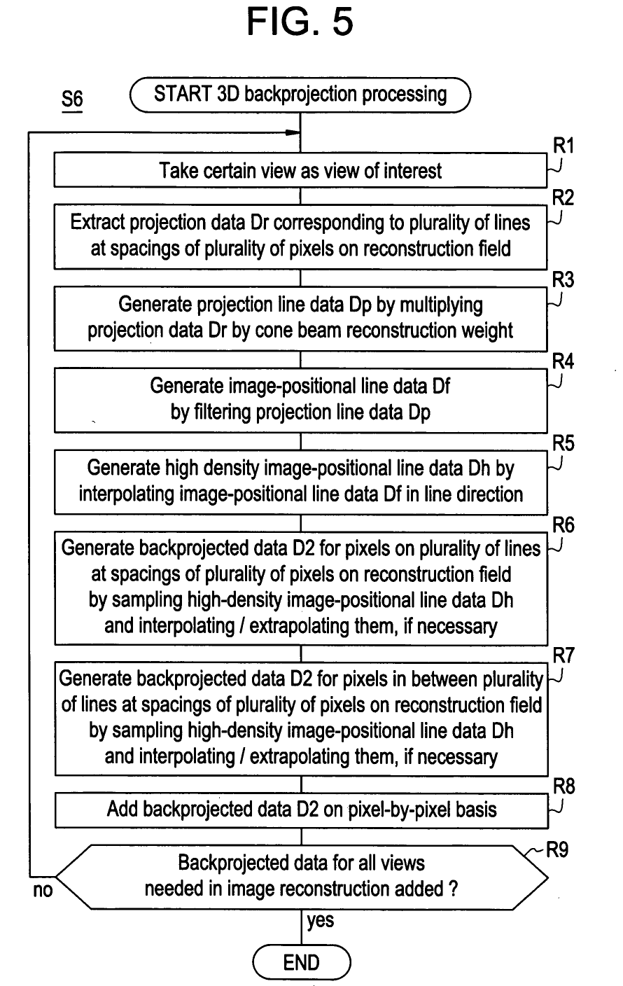

At Step S4, the filtered projection data D0(z, view, j, i) is subjected to three-dimensional backprojection processing to determine backprojected data D3(x, y). The three-dimensional backprojection processing will be discussed below with reference to FIG. 5. At Step S5, the backprojected data D3(x, y) is subjected to post-processing to obtain a CT image.

FIG. 5 is a flow chart showing details of the three-dimensional backprojection processing (Step S4 in FIG. 4). The flow chart represents an operation of the central processing apparatus 3. At Step R1, one view is taken as a view of interest from among all views needed in reconstruction of a CT image (i.e., views for 360° or for “180°+fan angle”). At Step R2, projection data Dr corresponding to a plurality of parallel lines at spacings of a plurality of pixels on a reconstruction field are extracted from among the projection data D0(z, view, j, i) at the view of interest.

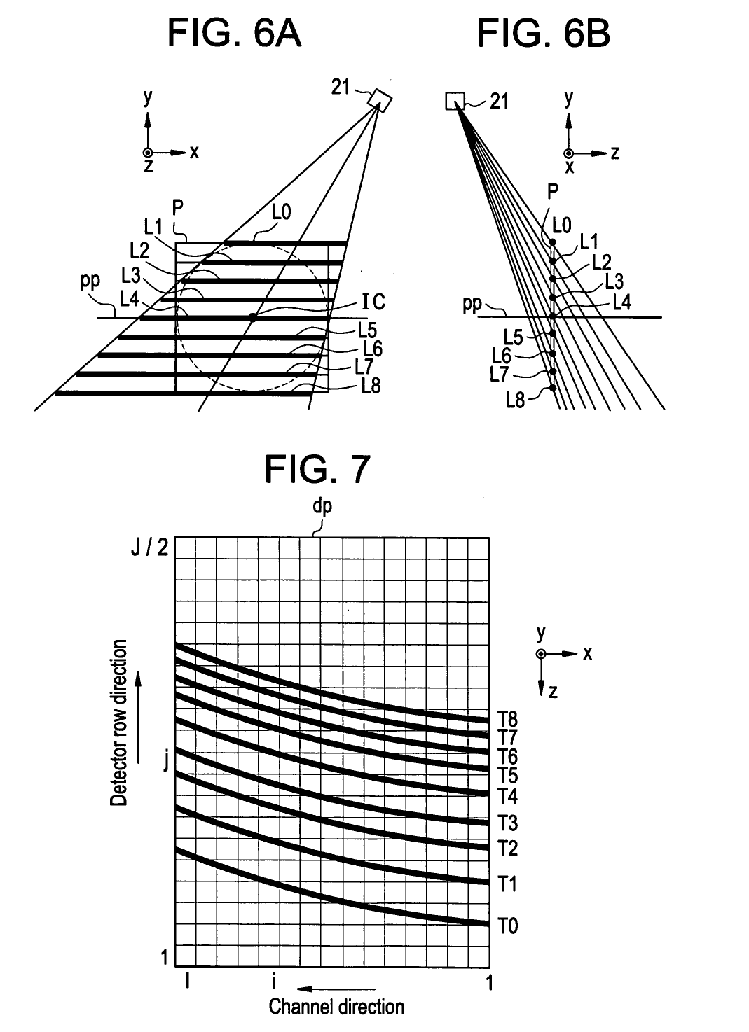

FIG. 6 exemplarily shows a plurality of parallel lines L0-L8 on the reconstruction field P. The number of lines is 1/64-½ of the maximum number of pixels in the reconstruction field in a direction orthogonal to the lines. For example, if the number of pixels in the reconstruction field P is 512×512, the number of lines is nine.

Moreover, the line direction is defined as the x-direction for −45°≦rotation angle<45° (or a view angle range mainly including the range and also including its vicinity) and 135°<rotation angle<225° (or a view angle range mainly including the range and also including its vicinity). The line direction is defined as the y-direction for 45°<rotation angle<135° (or a view angle range mainly including the range and also including its vicinity) and 225°≦rotation angle<315° (or a view angle range mainly including the range and also including its vicinity). Furthermore, a projection plane pp is assumed to pass through the center of rotation IC and be parallel to the lines L0-L8.

FIG. 7 shows lines T0-T8 formed by projecting the plurality of parallel lines L0-L8 onto a detector plane dp in the direction of X-ray transmission. The direction of X-ray transmission is determined depending upon the geometrical position of the X-ray tube 21, multi-row detector 24 and lines L0-L8 (including the distance in the z-axis direction from the x-y plane passing through the center in the z-axis direction of the multi-row detector 24 to the image reconstruction field P, and the positions of the lines L0-L8 formed by a set of pixel points on the image reconstruction plane P); since the position z of the projection data D0(z, view, j, i) in the direction of the table's rectilinear motion is known, the direction of X-ray transmission can be accurately determined.

The projection data Dr corresponding to the lines L0-L8 can be obtained by extracting projection data at the detector row j and channel i corresponding to the lines T0-T8 projected onto the detector plane dp. The projection data Dr is obtained by interpolation or extrapolation, if necessary.

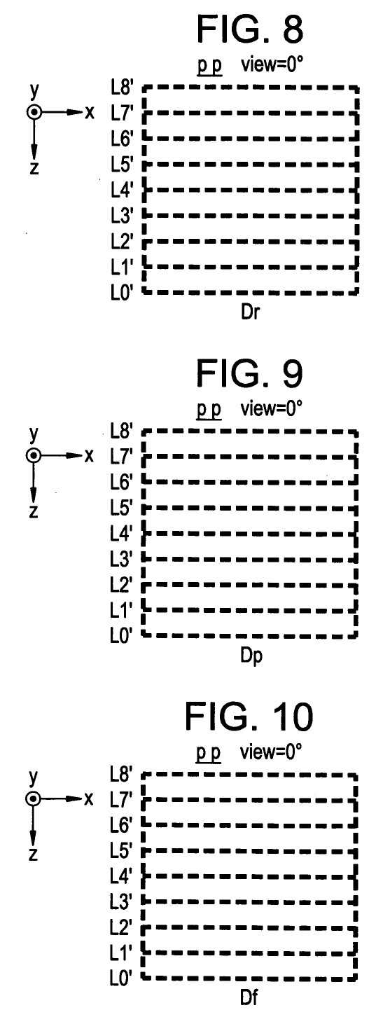

Now lines L0′-L8′ formed by projecting the lines T0-T8 onto the projection plane pp in the direction of X-ray transmission are assumed as shown in FIG. 8, and the projection data Dr are arranged over the lines L0′-L8′ based on the z-axis coordinate information.

Referring again to FIG. 5, at Step R3, the projection data Dr of the lines L0′-L8′ are multiplied by a cone beam reconstruction weight to generate projection line data Dp as shown in FIG. 9. The cone beam reconstruction weight is (r1/r0)², where r0 is the distance from the focal spot of the X-ray tube 21 to the j-th detector row and the i-th channel of the multi-row detector 24 corresponding to projection data Dr, and r1 is the distance from the focal spot of the X-ray tube 21 to a point on the reconstruction field corresponding to the projection data Dr.

At Step R4, the projection line data Dp are filtered. Specifically, the projection line data Dp are subjected to FFT, multiplied by a filter function (reconstruction function), and subjected to inverse FFT to generate image-positional line data Df as shown in FIG. 10.

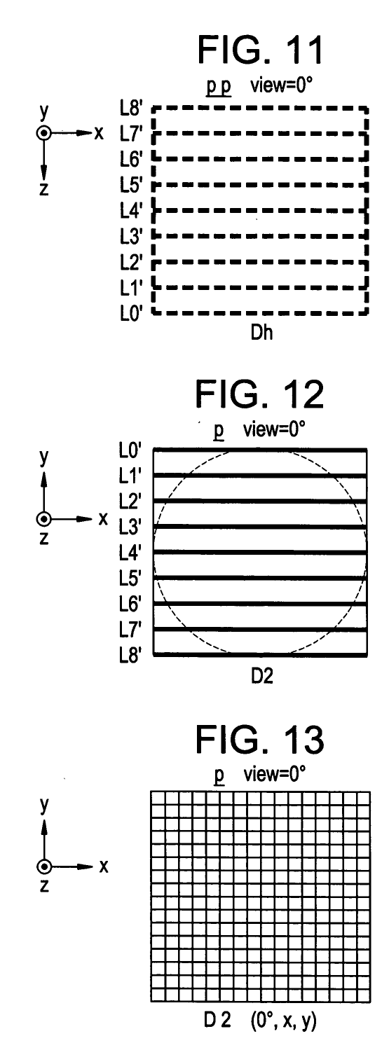

At Step R5, the image-positional line data Df is interpolated in the line direction to generate high-density image-positional line data Dh as shown in FIG. 11. The data density of the high-density image-positional line data Dh is 8-32 times the maximum number of pixels in the reconstruction field in the line direction. For example, if the factor is 16 and the number of pixels in the reconstruction field P is 512×512, the data density is 8,192 points/line. The central processing apparatus 3 conducting the processing from Step R1 to R5 is an example. At Step R6, the high-density image-positional line data Dh are sampled and interpolated/extrapolated, if necessary, to generate backprojected pixel data D2 for pixels on the lines L0-L8, as shown in FIG. 12.

At Step R7, the high-density image-positional line data Dh are sampled and interpolated/extrapolated to generate backprojected pixel data D2 for pixels in between the lines L0-L8, as shown in FIG. 13. The central processing apparatus 3 conducting the processing from Step R6 to R7 is an example of the backprojected pixel data calculating means of the present invention.

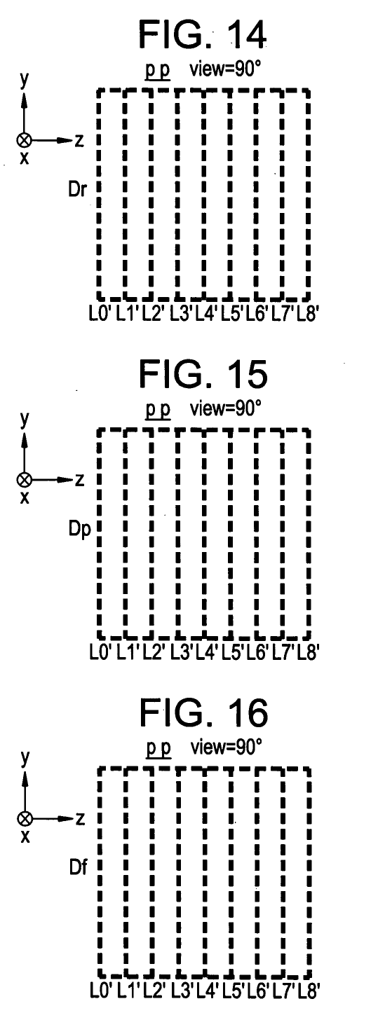

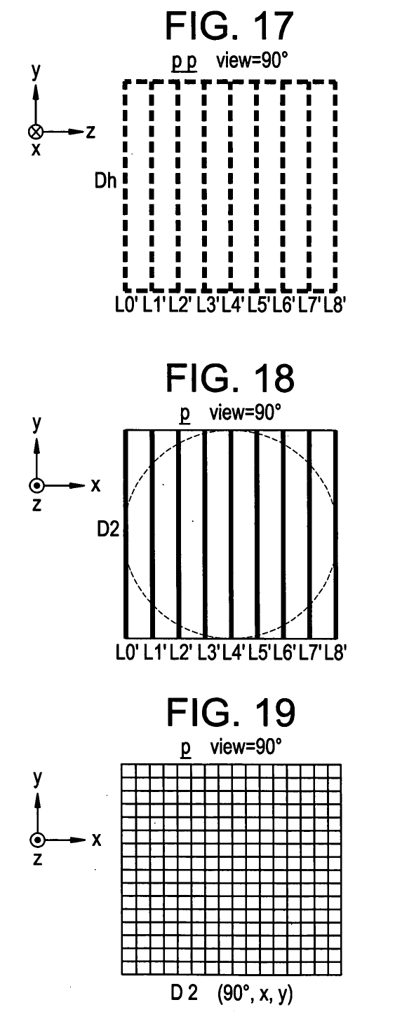

In FIGS. 8-13, −45°≦rotation angle<45° (or a view angle range mainly including the range and also including its vicinity) and 135°≦rotation angle<225° (or a view angle range mainly including the range and also including its vicinity) are assumed, while FIGS. 14-19 are applied for 45°≦rotation angle<135° (or a view angle range mainly including the range and also including its vicinity) and 225°≦rotation angle<315° (or a view angle range mainly including the range and also including its vicinity).

The backprojected pixel data D2 may be determined as the result of weighted addition on backprojected pixel data D2 at a certain rotation angle (view) and backprojected pixel data D2 at an opposite rotation angle (view) with weighting factors wₐ and w_{b} (wₐ+w_{b}=1) that depend upon the angle formed by a straight line connecting a pixel on the reconstruction field at these views and the X-ray focal spot with respect to the plane of the reconstruction field.

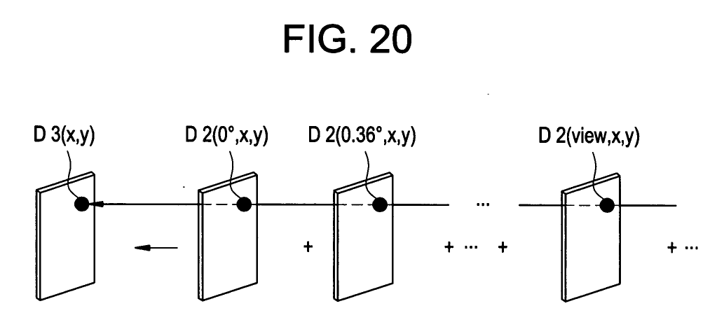

Referring again to FIG. 5, at Step R8, the backprojected pixel data D2 shown in FIG. 13 or 19 are added on a pixel-by-pixel basis, as shown in FIG. 20. At Step R9, Steps R1-R8 are repeated for all views needed in reconstruction of a CT image (i.e., views for 360° or for “180°+fan angle”) to obtain backprojected data D3(x, y). The central processing apparatus 3 conducting the processing from Step R8 to R9 is an example of the backprojected data calculating means of the present invention.

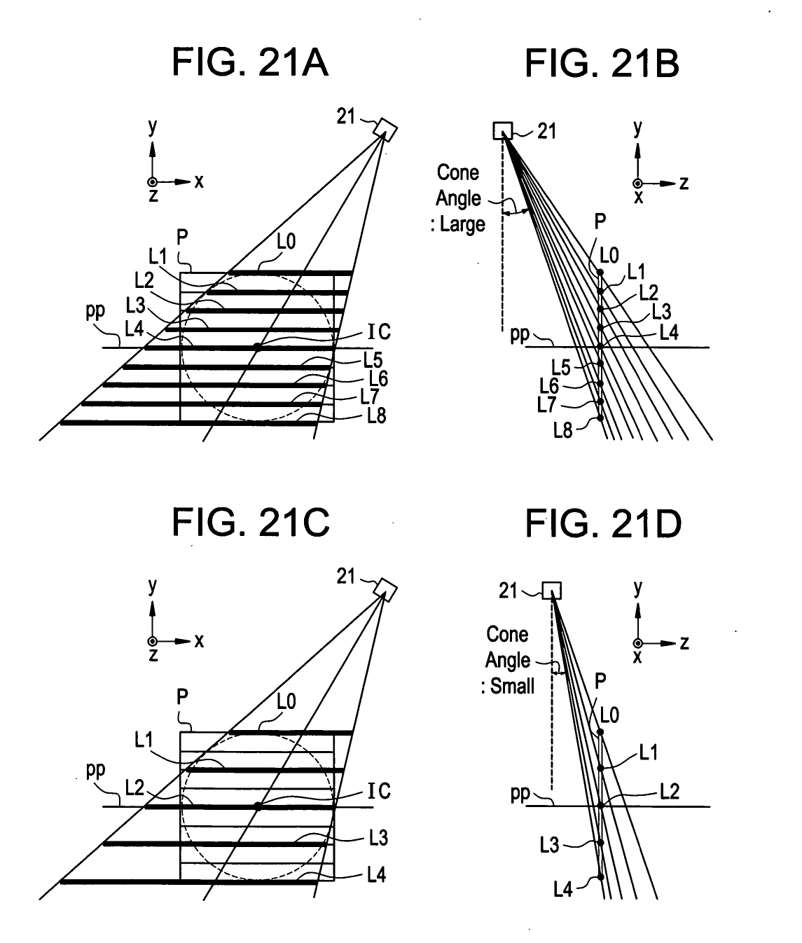

According to the X-ray CT apparatus 100, as shown in FIG. 21, when the spacing (indicated by gray bold lines in the drawing) between the planar projection lines on the projection plane pp is to be kept constant for keeping image quality, a larger number of planar projection lines is selected to keep the spacing between the planar projection lines for a larger cone angle formed between the reconstruction plane (X-Y plane) and X-ray beam. For a smaller cone angle, the spacing between the planar projection lines can be kept even if the number of planar projection lines is small.

In other words, for a smaller cone angle formed between the reconstruction plane (X-Y plane) and X-ray beam, the number of planar projection lines may be decreased to reduce the time for reconstruction relative to the case of a larger cone angle. Specifically, for rows near the center in the X-ray multi-row detector, the number of planar projection lines may be decreased to achieve image reconstruction by three-dimensional backprojection at a high speed, and for rows near the edges in the X-ray multi-row detector, the number of planar projection lines may be increased to an extent that does not degrade image quality, thereby reducing the time for image reconstruction processing by three-dimensional backprojection.

Since the reconstruction field around the rows near the edges in the X-ray multi-row detector has some tolerance regarding the signal S/N as compared with the rows near the center, an appropriate filter may be used to attenuate X-rays around the rows near the edges in the X-ray multi-row detector to obtain a uniform S/N. This can also reduce the exposure dose to the subject.

Moreover, different reconstruction kernels may be employed between the rows near the center and those near the edges in the X-ray multi-row detector. In this case, a low-emphasis kernel is used for the rows near the center, and a high-emphasis kernel is used for the rows near the edges. This can also provide a uniform S/N.

Furthermore, the uniform S/N may be obtained by differentiating the number of data elements involved in interpolation between the rows near the center and those near the edges. In this case, the number of data elements involved in interpolation is increased for the rows near the center, and is decreased for the rows near the edges.

The technique for image reconstruction may be a conventionally known three-dimensional image reconstruction technique according to the Feldkamp method. Moreover, three-dimensional image reconstruction techniques proposed in Japanese Patent Application Nos. 2002-066420, 2002-147061, 2002-147231, 2002-235561, 2002-235662, 2002-267833, 2002-322756 and 2002-338947 may be employed.

Many widely different embodiments of the invention may be configured without departing from the spirit and the scope of the present invention. It should be understood that the present invention is not limited to the specific embodiments described in the specification, except as defined in the appended claims.

1. A three-dimensional backprojection method comprising the steps of:
plane-projecting projection data D0 collected by an axial scan using a multi-row X-ray detector or planar X-ray detector having a plurality of detectors, onto a projection plane to determine plane-projected data D1;
then projecting said plane-projected data D1 in a direction of X-ray transmission onto pixels constituting a plurality of lines arranged successively in a direction parallel to the projection plane at spacings of a plurality of pixels on a reconstruction field, to determine backprojected pixel data D2 for pixels constituting lines on the reconstruction field for a number of plane-projected data lines that depends upon the angle formed between the plane of the reconstruction field and X-ray beam;
interpolating said plurality of lines to determine backprojected pixel data D2 for pixels in between the lines on the reconstruction field; and
adding the backprojected pixel data D2 on a pixel-by-pixel basis for all views used in image reconstruction to determine backprojected data D3.

2. The three-dimensional backprojection method of claim 1, wherein the number of plane-projected data lines is optimized taking image quality of an image to be reconstructed into account.

3. The three-dimensional backprojection method of claim 1, wherein: representing a direction perpendicular to a plane of rotation of an X-ray tube and an X-ray detector as z-direction, a direction of the center axis of the X-ray beam at a rotation angle of 0° as y-direction, and a direction orthogonal to the z- and y-directions as x-direction, said projection plane is defined as the x-z plane that passes through a center of rotation for −45°≦rotation angle<45° or a rotation angle range mainly including the range and also including its vicinity and 135°≦rotation angle<225° or a rotation angle range mainly including the range and also including its vicinity, and said projection plane is defined as the y-z plane that passes through the center of rotation for 45°≦rotation angle<135° or a rotation angle range mainly including the range and also including its vicinity and 225°≦rotation angle<315° or a rotation angle range mainly including the range and also including its vicinity.

4. The three-dimensional backprojection method of claim 1, wherein each data element of the plane-projected data D1 is determined from a plurality of data elements of the projection data D0 by extrapolation.

5. The three-dimensional backprojection method of claim 1, wherein each data element of the backprojected pixel data D2 is determined by weighted addition on a plurality of data elements of the plane-projected data D1.

6. The three-dimensional backprojection method of claim 1, wherein the backprojected pixel data D2 is determined as the result of weighted addition on backprojected pixel data D2 at a certain rotation angle (view) and backprojected pixel data D2 at an opposite rotation angle (view) with weighting factors wₐ and w_{b} (wₐ+w_{b}=1) that depend upon the angle formed by a straight line connecting a pixel on the reconstruction field at these views and an X-ray focal spot with respect to the plane of the reconstruction field.

7. A three-dimensional backprojection apparatus comprising:
a plane-projected data calculating device for plane-projecting projection data D0 collected by an axial scan using a multi-row X-ray detector or planar X-ray detector having a plurality of detectors, onto a projection plane to determine plane-projected data D1;
a backprojected pixel data calculating device for projecting said plane-projected data D1 in a direction of X-ray transmission onto pixels constituting a plurality of lines arranged successively in a direction parallel to the projection plane at spacings of a plurality of pixels on a reconstruction field, to determine backprojected pixel data D2 for pixels constituting lines on the reconstruction field for a number of plane-projected data lines that depends upon the angle formed between the plane of the reconstruction field and X-ray beam, and for interpolating in between said plurality of lines to determine backprojected pixel data D2 for pixels in between the lines on the reconstruction field; and
a backprojected data calculating device for adding the backprojected pixel data D2 on a pixel-by-pixel basis for all views used in image reconstruction to determine backprojected data D3.

8. The three-dimensional backprojection apparatus of claim 7, wherein the number of plane-projected data lines is optimized taking image quality of an image to be reconstructed into account.

9. The three-dimensional backprojection apparatus of claim 7, wherein: representing a direction perpendicular to a plane of rotation of an X-ray tube and an X-ray detector as z-direction, a direction of the center axis of the X-ray beam at a rotation angle of 0° as y-direction, and a direction orthogonal to the z- and y-directions as x-direction, said plane-projected data calculating device defines said projection plane as the x-z plane that passes through a center of rotation for −45°≦rotation angle<45° or a rotation angle range mainly including the range and also including its vicinity and 135°≦rotation angle<225° or a rotation angle range mainly including the range and also including its vicinity, and defines said projection plane as the y-z plane that passes through the center of rotation for 45°≦rotation angle<135° or a rotation angle range mainly including the range and also including its vicinity and 225°≦rotation angle<315° or a rotation angle range mainly including the range and also including its vicinity.

10. The three-dimensional backprojection apparatus of claim 7, wherein: said plane-projected data calculating device determines each data element of the plane-projected data D1 from a plurality of data elements of the projection data D0 by extrapolation.

11. The three-dimensional backprojection apparatus of claim 7, wherein said backprojected pixel data calculating device determines each data element of the backprojected pixel data D2 by weighted addition on a plurality of data elements of the plane-projected data D1.

12. The three-dimensional backprojection apparatus of claim 7, wherein said backprojected pixel data calculating device determines the backprojected pixel data D2 as the result of weighted addition on backprojected pixel data D2 at a certain rotation angle (view) and backprojected pixel data D2 at an opposite rotation angle (view) with weighting factors wₐ and w_{b} (wₐ+w_{b}=1) that depend upon the angle formed by a straight line connecting a pixel on the reconstruction field at these views and an X-ray focal spot with respect to the plane of the reconstruction field.

13. An X-ray CT apparatus comprising:
an X-ray tube;
a multi-row detector having a plurality of detector rows;
a scanning device for collecting projection data D0 while rotating at least one of said X-ray tube and said multi-row detector around a subject to be imaged;
a plane-projected data calculating device for determining plane-projected data D1 plane-projected onto a projection plane based on said projection data D0;
a backprojected pixel data calculating device for projecting said plane-projected data D1 in a direction of X-ray transmission onto pixels constituting a plurality of lines arranged successively in a direction parallel to the projection plane at spacings of a plurality of pixels on a reconstruction field, to determine backprojected pixel data D2 for pixels constituting lines on the reconstruction field for a number of plane-projected data lines that depends upon the angle formed between the plane of the reconstruction field and X-ray beam, and for interpolating in between said plurality of lines to determine backprojected pixel data D2 for pixels in between the lines on the reconstruction field; and
a backprojected data calculating device for adding the backprojected pixel data D2 on a pixel-by-pixel basis for all views used in image reconstruction to determine backprojected data D3.

14. The X-ray CT apparatus of claim 13, wherein the number of plane-projected data lines is optimized taking image quality of an image to be reconstructed into account.

15. The X-ray CT apparatus of claim 13, wherein: representing a direction perpendicular to a plane of rotation of the X-ray tube and X-ray detector as z-direction, a direction of the center axis of the X-ray beam at a rotation angle of 0° as y-direction, and a direction orthogonal to the z- and y-directions as x-direction, said plane-projected data calculating device defines said projection plane as the x-z plane that passes through a center of rotation for −45°≦rotation angle<45° or a rotation angle range mainly including the range and also including its vicinity and 135°≦rotation angle<225° or a rotation angle range mainly including the range and also including its vicinity, and defines said projection plane as the y-z plane that passes through the center of rotation for 45°≦rotation angle<135° or a rotation angle range mainly including the range and also including its vicinity and 225°≦ rotation angle<315° or a rotation angle range mainly including the range and also including its vicinity.

16. The X-ray CT apparatus of claim 13, wherein said plane-projected data calculating device determines each data element of the plane-projected data D1 from a plurality of data elements of the projection data D0 by extrapolation.

17. The X-ray CT apparatus of claim 13, wherein said backprojected pixel data calculating device determines each data element of the backprojected pixel data D2 by weighted addition on a plurality of data elements of the plane-projected data D1.

18. The X-ray CT apparatus of claim 13, wherein said backprojected pixel data calculating device determines the backprojected pixel data D2 as the result of weighted addition on backprojected pixel data D2 at a certain rotation angle (view) and backprojected pixel data D2 at an opposite rotation angle (view) with weighting factors wₐ and w_{b} (wₐ+w_{b}=1) that depend upon the angle formed by a straight line connecting a pixel on the reconstruction field at these views and an X-ray focal spot with respect to the plane of the reconstruction field.
